(19) United States
(12) Patent Application Publication
(10) Pub. No.: US 2020/0323555 A1
(43) Pub. Date: Oct. 15, 2020

(54) ENDOSCOPIC CANNULA

(71) Applicant: Wuhan Youcare Technology Co., Ltd., Wuhan (CN)

(72) Inventors: Gang LONG, Wuhan (CN); Jinping LI, Wuhan (CN); Yeyun MAO, Wuhan (CN); Glenn Michael PREMINGER, Wuhan (CN); Zhangqun YE, Wuhan (CN); Jianxing LI, Wuhan (CN); Xiao YU, Wuhan (CN); Junhui ZHANG, Wuhan (CN); He XIAO, Wuhan (CN); Wencheng LI, Wuhan (CN); Xuecheng HU, Wuhan (CN); Yaohui WU, Wuhan (CN)

(21) Appl. No.: 16/784,224

(22) Filed: Feb. 6, 2020

Related U.S. Application Data
(63) Continuation-in-part of application No. PCT/CN2017/096214, filed on Aug. 7, 2017.

(51) Int. Cl. A61B 17/34; A61B 17/00

(52) U.S. Cl. CPC A61B 17/3423; A61B 17/3403; A61B 17/00234; A61B 2217/005; A61B 2017/00296; A61B 2017/00323; A61B 2017/3405; A61B 2017/00287

(57) ABSTRACT

An endoscopic cannula, including: a handle; a sheath; a core rod disposed in the sheath and capable of sliding along an axial direction of the sheath. The core rod includes a first channel, a second channel, and a third channel. The handle includes a first joint configured to guide a lead wire into the first channel, a second joint configured to guide water into the second channel, and a third joint configured to guide an endoscope into the third channel. The sheath includes a first end and a second end, and the second end is disposed in the handle.

### CROSS-REFERENCE TO RELATED APPLICATIONS

This application is a continuation-in-part of International Patent Application No. PCT/CN2017/096214 with an international filing date of Aug. 7, 2017, designating the United States, now pending, the contents of which, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND

The disclosure relates to an endoscopic cannula.

A cannula is a tube that can be inserted into the body for the delivery or removal of fluid. In simple terms, a cannula can surround the inner or outer surfaces of a trocar needle thus extending the effective needle length by at least half the length of the original needle. Conventional endoscope cannula for ureteroscopy is flexible. This leads to the difficulty of moving forward when encountering obstacles. As a result, the flexible cannula may not be able to reach the pathological site.

### SUMMARY

The disclosure provides an endoscopic cannula that comprises a hard sheath or a hard sheath and a soft sheath.

Provided is an endoscopic cannula, comprising: a handle; a sheath; a core rod disposed in the sheath and capable of sliding along an axial direction of the sheath. The core rod comprises a first channel, a second channel, and a third channel which are sequentially connected to one another. The handle comprises a first joint configured to guide a lead wire into the first channel, a second joint configured to guide water into the second channel, and a third joint configured to guide an endoscope into the third channel. The sheath comprises a first end and a second end, and the second end is disposed in the handle.

The sheath is a hard sheath; the hard sheath comprises a first end exposed out of the handle and a second end disposed in the handle; the hard sheath comprises an axial straight hole, and the core rod is in clearance fit with the axial straight hole.

The sheath is a combination of a soft sheath and a hard sheath; the hard sheath comprises a first end exposed out of the handle and a second end disposed in the handle; the soft sheath comprises a first end and a second end, and the second end of the soft sheath is coaxial and integrated with the first end of the hard sheath; an inner diameter and an outer diameter of the soft sheath are the same as that of the hard sheath; the soft sheath and the hard sheath each comprise an axial straight hole, and the core rod is in clearance fit with the axial straight hole; the soft sheath is provided with a steering wire, and the handle comprises a steering mechanism on which the steering wire is fixed.

The first end of the soft sheath is provided with a locking cap; the locking cap comprises a metal material and is coaxial with the soft sheath; one end of the steering wire is fixed on the locking cap, and the other end of the steering wire passes through the soft sheath and the hard sheath to fixedly connect to the steering mechanism.

One side of the handle comprises a hand shank; the steering mechanism is disposed on the hand shank; and the hand shank comprises a plurality of grooves.

The handle comprises a sleeve; the sleeve comprises a first end, a second end, and an axial through hole disposed between the first end and the second end; the axial through hole comprises a first section, a second section, and a third section; the hard sheath sequentially passes through the first section, the second section, and the third section and is fixed on the sleeve; the third section is a taper hole; the second end of the sleeve is provided with a location column, and the first joint, the second joint and the third joint are disposed on the location column.

The location column comprises a casing; the casing comprises a counter bore configured to receive the core rod; the counter bore comprises a first hole communicating with the first channel, a second hole communicating with the second channel, and a third hole communicating with the third channel; the first joint, the second joint and the third joint communicate with the first hole, the second hole, and the third hole, respectively; two sides of the casing each are provided with a connection plate and a fastener; two sides of the second end of the sleeve each are provided with a locating slot; and the locating slot matches the fastener in shape to lock the sleeve and the casing.

The second end of the sleeve is provided with a disc; the disc is coaxial with the sleeve and comprises a via hole communicating and coaxial with the third section of the axial through hole; the disc comprises a circumferential flange, and the locating slot is disposed on the circumferential flange.

Two connection rods are connected to the circumferential flange, and one end of each of the two connection rods is provided with a string bag for accommodating broken stones.

One side of the sleeve is provided with a suction channel; the suction channel comprises a first opening communicating with the third section of the axial through hole and a second opening configured to connect to a vacuum machine; the first opening of the suction channel is disposed between the via hole and the first section of the axial through hole.

The first channel shares one channel with the second channel, and the third channel is an independent channel.

The first channel, the second channel, and the third channel of the core rod are independent from each other.

The hard sheath and/or the soft sheath a head end provided with a plurality of circular holes.

Advantages of the endoscopic cannula according to embodiments of the disclosure are summarized as follows. As a transitional guiding device, the core rod can be used in the urological surgery with existing endoscopes to guide the ureteroscope to reach the operative site accurately. Specifically, the core rod and the sheath are guided by the lead wire to the operative site. The ureter can be observed under the endoscope, and when the ureter is blocked, the water can be injected into the axial through hole to remove the obstacle. When the core rod and the sheath reach the operative site, the core rod is withdrawn, a ureteroscope is inserted in the sheath to perform flexible ureteroscopy. During the operation, the position of the ureteroscope can be adjusted by turning the steering mechanism, so that the soft and hard sheath can enter the renal pelvis and calyces and can be positioned. The ureteroscope, together with the operation instrument, breaks the stones in the renal pelvis and calyces. A vacuum machine is connected to the suction channel of the handle. When the broken stones are observed, the vacuum machine is started, and the front end of the ureteroscope exits from the soft sheath. The negative pressure in the soft sheath will suck the broken stones out of the body. The broken stones are placed in the string bag. After the operation, the broken stones in the string bag are treated. Based on the endoscopic cannula comprising the core rod and the location column of the disclosure, the ureteroscope can move to the operative site accurately. The operation is easy and convenient, can reduce the amount of bleeding and the pain of the patients, and improves the operation efficiency and success rate.

### BRIEF DESCRIPTION OF THE DRAWINGS

FIG. 1 is a stereogram of an endoscopic cannula comprising a hard sheath according to one embodiment of the disclosure;

FIG. 2 is a cooperative diagram of a hard sheath and a location column according to one embodiment of the disclosure;

FIG. 3 is a front view of an endoscopic cannula comprising a hard sheath according to one embodiment of the disclosure;

FIG. 4 is a sectional view taken from line A-A in FIG. 3;

FIG. 5 is a side view of an endoscopic cannula comprising a hard sheath according to one embodiment of the disclosure;

FIG. 6 is a sectional view taken from line B-B in FIG. 5;

FIG. 7 is a stereogram of an endoscopic cannula comprising a hard sheath and a soft sheath according to another embodiment of the disclosure;

FIG. 8 is a front view of an endoscopic cannula comprising a hard sheath and a soft sheath according to another embodiment of the disclosure;

FIG. 9 is a sectional view taken from line C-C in FIG. 8;

FIG. 10 is a side view of an endoscopic cannula comprising a hard sheath and a soft sheath according to another embodiment of the disclosure;

FIG. 11 is a sectional view taken from line D-D in FIG. 10;

FIG. 12 is a front view of a location column according to one embodiment of the disclosure;

FIG. 13 is a sectional view taken from arrow K in FIG. 12;

FIG. 14 is a front view of a core rod according to one embodiment of the disclosure;

FIG. 15 is a top view of a core rod according to one embodiment of the disclosure;

FIG. 16 is a front view of a handle according to one embodiment of the disclosure;

FIG. 17 is a sectional view taken from line E-E in FIG. 16; and

FIG. 18 is a schematic diagram of a disc according to one embodiment of the disclosure.

In the drawings, the following reference numbers are used: 1. Handle; 1.1. Sleeve; 1.2. Disc; 1.3. Hand shank; 2. Sheath; 2.1. Hard sheath; 2.2. Soft sheath; 3. Core rod; 4. Location column; 4.1. Casing; 4.2. Counter bore; 5. First channel; 6. Second channel; 7. Third channel; 8. First joint; 9. Second joint; 10. Third joint; 11. Steering wire; 12. Steering mechanism; 13. First section; 14. Third section; 15. Locating slot; 16. First hole; 17. Second hole; 18. Third hole; 19. Fastener; 20. Locking cap; 21. Via hole; 22. Circumferential flange; 23. Connection rod; 24. String bag; 25. First opening; 26. Groove; 27. Connection plate; 28. Location blocks; 29. Groove; 30. Second opening; 31. Seal plug; 32. Second section.

### DETAILED DESCRIPTION OF THE EMBODIMENTS

To further illustrate, embodiments detailing an endoscopic cannula are described below. It should be noted that the following embodiments are intended to describe and not to limit the disclosure.

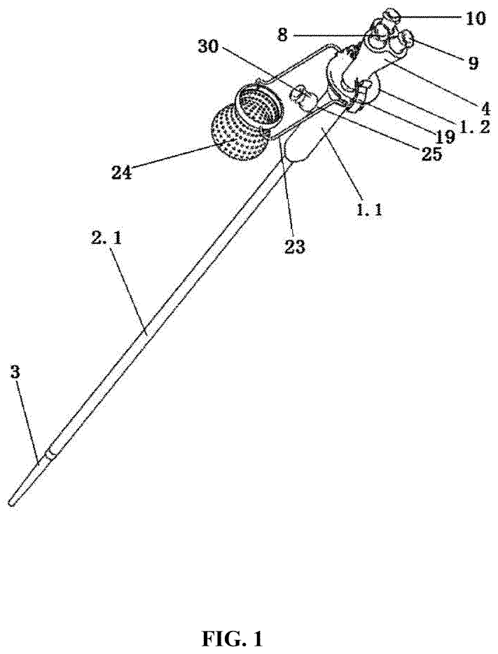

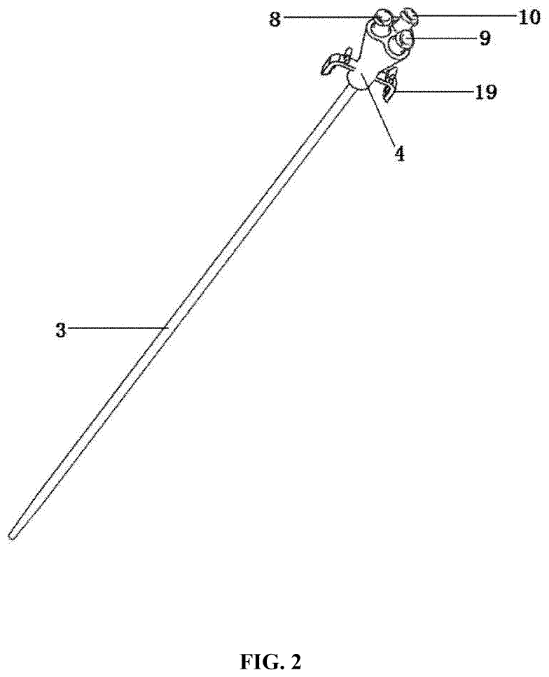

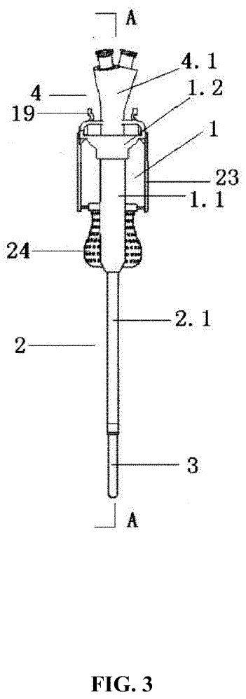

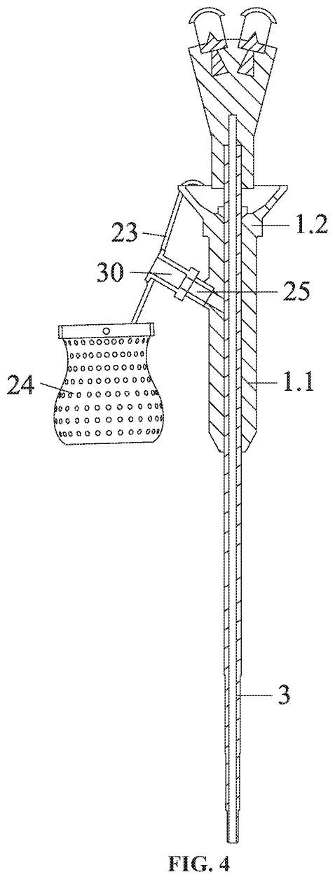

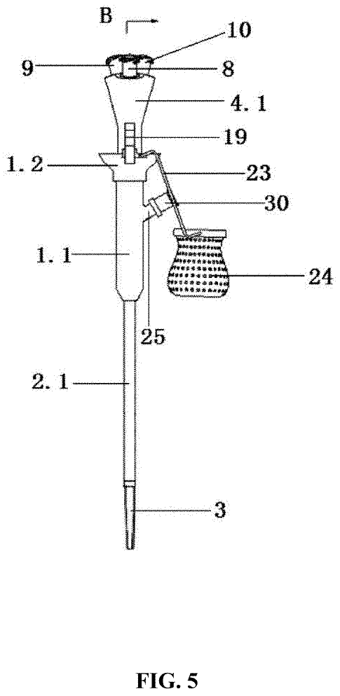

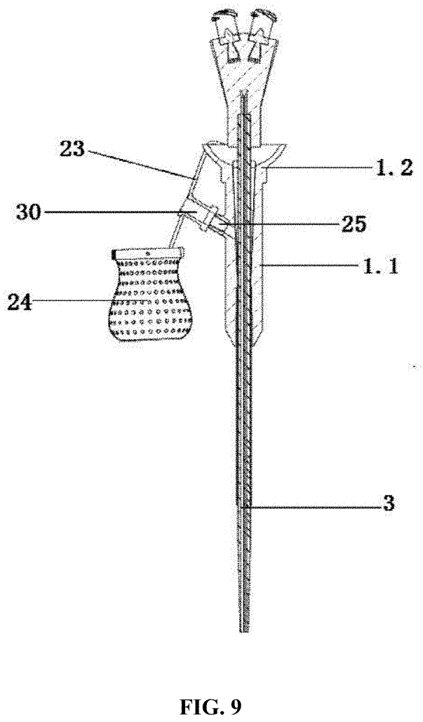

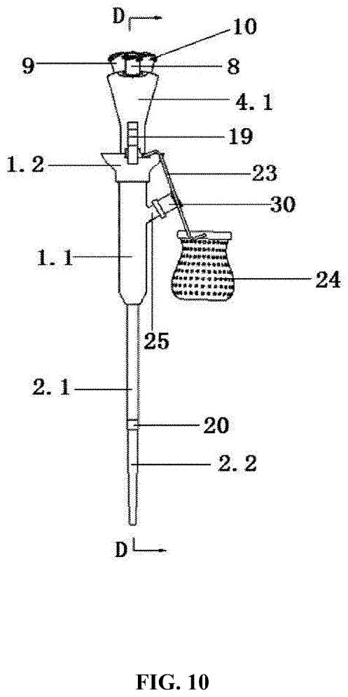

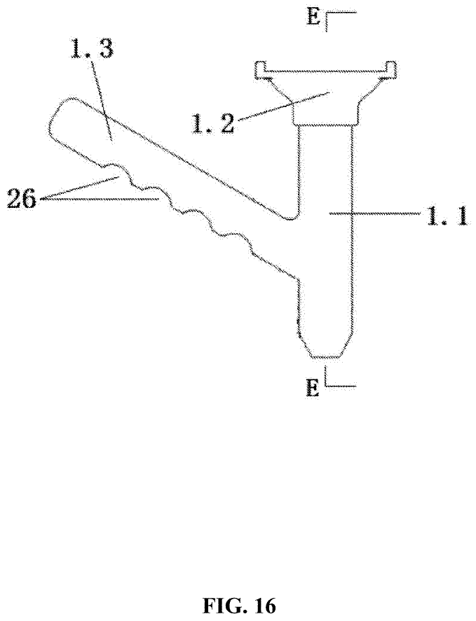

FIGS. 1-18 show an endoscopic cannula for urological operations. The endoscopic cannula comprises a handle 1; a sheath 2; a core rod 3 disposed in the sheath 2 and capable of sliding along an axial direction of the sheath 2. The cord rod 3 is made of is medical polymer material (but not limited to polymer material), which has certain plasticity and can deform and bend, so that the cord rod can enter the human body and reach the pathological part conveniently.

The sheath 2 is a hard sheath 2.1 or a combination of a soft sheath 2.2 and a hard sheath 2.1.

As shown in FIGS. 1-6 and FIG. 17, the hard sheath 2.1 comprises a first end exposed out of the handle 1 and a second end disposed in the handle 1; the hard sheath 2.1 comprises an axial straight hole, and the core rod 3 is in clearance fit with the axial straight hole.

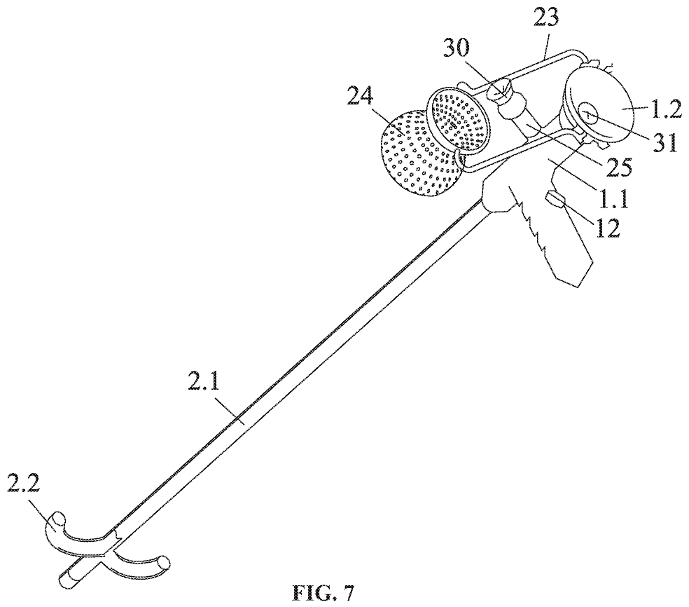

As shown in FIGS. 7-18, the sheath 2 is a combination of a soft sheath 2.2 and a hard sheath 2.1; the hard sheath 2.1 comprises a first end exposed out of the handle 1 and a second end disposed in the handle 1; the soft sheath 2.2 comprises a first end and a second end, and the second end of the soft sheath 2.2 is coaxial and integrated with the first end of the hard sheath; an inner diameter and an outer diameter of the soft sheath 2.2 are the same as that of the hard sheath 2.1; the soft sheath 2.2 and the hard sheath 2.1 each comprise an axial straight hole, and the core rod 3 is in clearance fit with the axial straight hole; the soft sheath 2.2 is provided with a steering wire 11, and the handle 1 comprises a steering mechanism 12 on which the steering wire 1 is fixed.

As shown in FIGS. 1-18, the second end of the sleeve 1.1 is provided with a location column 4. The handle 1 comprises a sleeve 1.1; the sleeve 1.1 comprises a first end, a second end, and an axial through hole disposed between the first end and the second end; the axial through hole comprises a first section 13, a second section 32, and a third section 14; the hard sheath 2.1 sequentially passes through the first section 13, the second section 32, and the third section 14 and is fixed on the sleeve 1.1; the third section 14 is a taper hole. The second end of the sleeve 1.1 is provided with a disc 1.2. One side of the sleeve 1.1 is provided with a hand shank 1.3 and a suction channel. The suction channel comprises a first opening 25 communicating with the third section 14 of the axial through hole and a second opening 30 configured to connect to a vacuum machine; the first opening 25 of the suction channel is disposed between the via hole 21 and the first section 13 of the axial through hole (so that the broken stone can enter the first opening 25 from the third section 14 of the axial through hole of the core rod). When the suction channel is not in use, a plug can be used to block the second opening 30, to avoid the internal pollution of the endoscope.

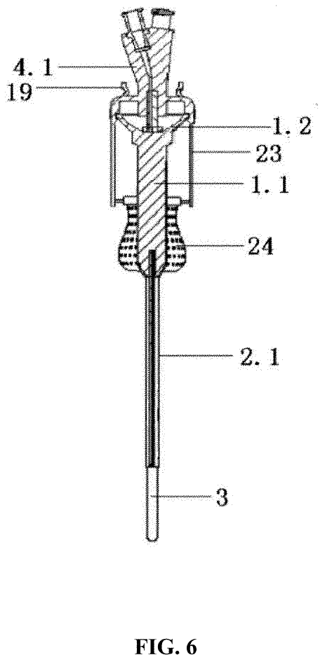

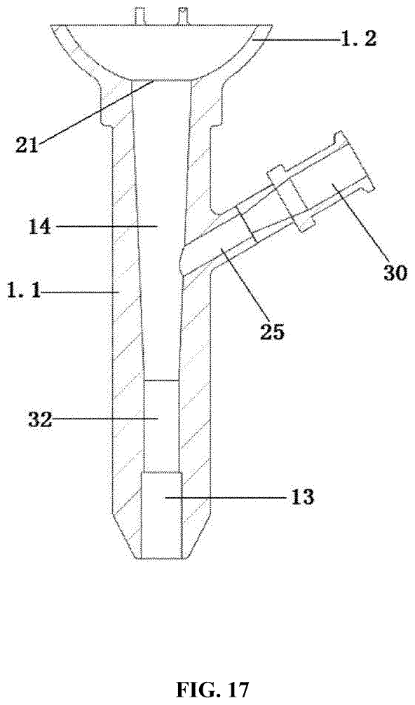

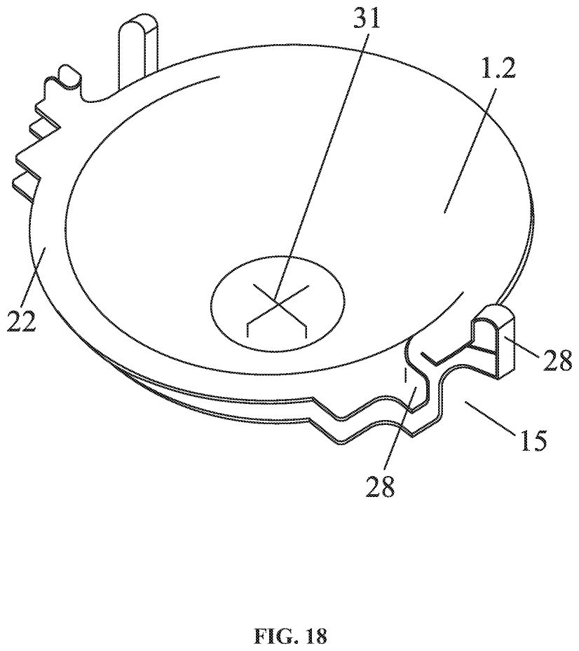

As shown in FIGS. 17-18, the disc 1.2 is coaxial with the sleeve 1.1 and comprises a via hole 21 communicating and coaxial with the third section 14 of the axial through hole (as shown in FIG. 17, when no core rod is inserted, the via hole 2 is blocked by a seal plug 31, to avoid the internal pollution of the endoscope). The disc 1.2 comprises a circumferential flange 22, and the locating slot 15 is disposed on the circumferential flange 22. As shown in FIGS. 6-10, two connection rods 23 are connected to the circumferential flange 22, and one end of each of the two connection rods 23 is provided with a string bag 24 for accommodating broken stones. The third section 14 of the axial through hole is the extension channel of the core rod 3 and the endoscope, and the endoscope enters the sheath 2 via the third joint 10.

As shown in FIGS. 7-11, the sheath 2 is a combination of a soft sheath 2.2 and a hard sheath 2.1; the hard sheath 2.1 comprises a first end exposed out of the handle 1 and a second end disposed in the first section 13 of the handle 1; the soft sheath 2.2 comprises a first end and a second end, and the second end of the soft sheath 2.2 is coaxial and integrated with the first end of the hard sheath. The soft sheath 2.2 and the hard sheath 2.1 each comprise an axial straight hole in clearance fit with the core rod 3. The first end of the soft sheath 2.2 is provided with a locking cap 20; the locking cap 20 is coaxial with the soft sheath 2.2; one end of the steering wire 11 is fixed on the locking cap 20, and the other end of the steering wire 11 passes through the soft sheath 2.2 and the hard sheath 2.1 to fixedly connect to the steering mechanism 12. The steering mechanism 12 is disposed on the hand shank 1.3; and the hand shank 1.3 comprises a plurality of grooves 26.

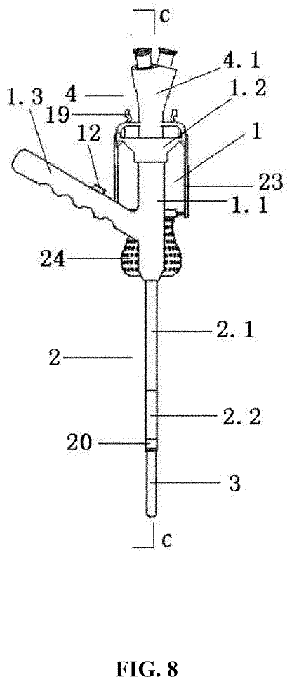

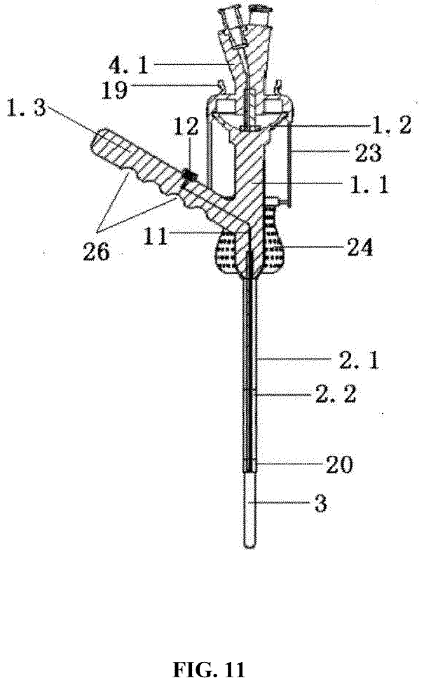

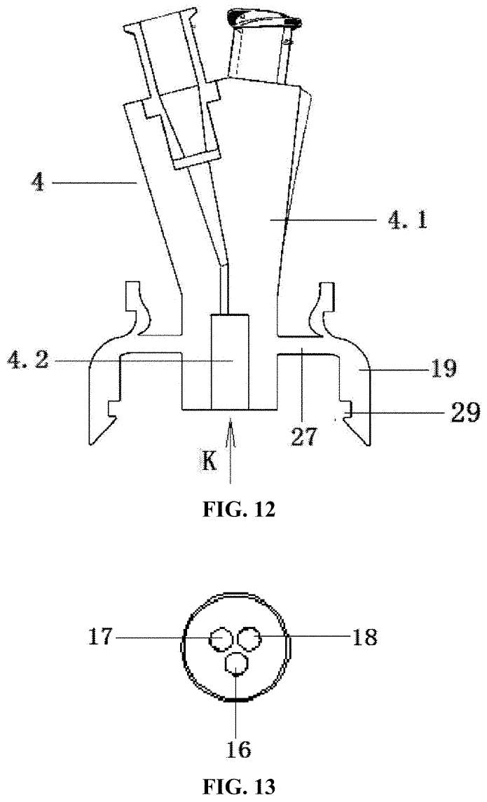

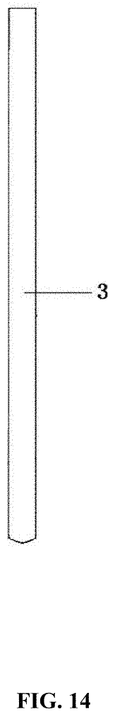

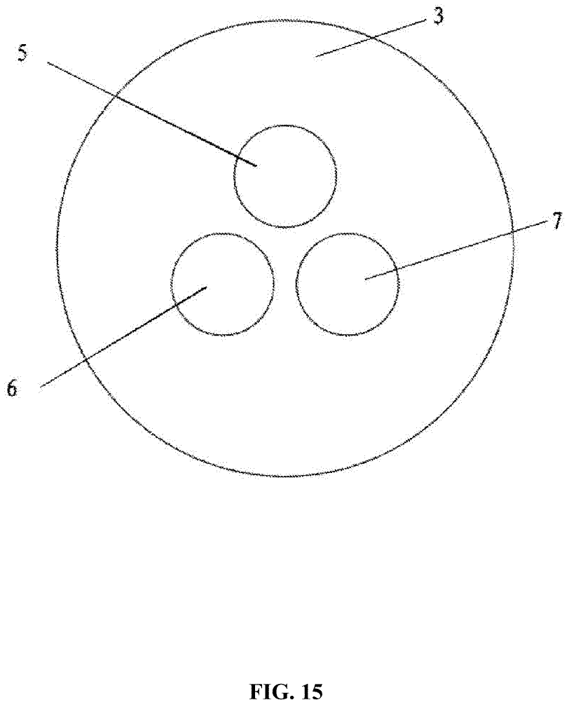

As shown in FIGS. 14-15, the core rod 3 comprises a first channel 5, a second channel 6, and a third channel 7 which are sequentially connected to one another. As shown in FIGS. 8-12, the location column 4 comprises a casing 4.1; the casing 4.1 comprises a counter bore 4.2 configured to receive the core rod 3. As shown in FIG. 7, the counter bore 4.2 comprises a first hole 16 communicating with the first channel 5, a second hole 17 communicating with the second channel 6, and a third hole 18 communicating with the third channel 7. The first joint 8, the second joint 9 and the third joint 10 communicate with the first hole 16, the second hole 17, and the third hole 18, respectively. The first channel 5, the second channel 6, and the third channel 7 are all disposed in the core rod 3 and all have the same bore diameters. The first joint 8, the second joint 9 and the third joint 10 all have the same size, and the front end of the third channel 7 is provided with glass to avoid the contamination of the endoscope entering the human body. The first joint 8 and the second joint 9 can be replaced by one another, which improves the use efficiency of the endoscope during the operation and reduces the operation time. As shown in FIG. 11, two sides of the casing 4.1 each are provided with a connection plate 27 and a fastener 19. As shown in FIGS. 12 and 18, the disc 1.2 comprises a circumferential flange 22, and the locating slot 15 is disposed on the circumferential flange 22. Two sides of the locating slot 15 are provided with location blocks 28, respectively. The gap between two location blocks 28 constitutes the location slot 15. The fastener 19 is fixed on the flange 22. Specifically, the fastener 19 comprises a groove 29, and the flange 22 is clamped in the groove 19. The core rod 3 is first positioned in the location column 4, and then the sheath 2 is installed along the lead wire. When the core rod 3 and the sheath 2 reach the operating position, the core rod and the location column 4 can be separated from the handle 1, thus achieving the detachment of the core rod 3. The arrangement of the location column 4 facilitates the positioning and detachment of the core rod 3, with the following advantages: in the process of installing the sheath, the ureter can be observed in real time, if being clogged, the obstacles can be removed instantly by water injection. Through the real-time observation, the position of the sheath can be accurately determined. After the sheath is in place, the core rod 3 is extracted, and the ureteroscopy is performed with an endoscope. The operation is performed under the endoscope, and the endoscope is moved forward in the hard and soft sheath to the renal pelvis and calyces. A suction machine is connected to the suction channel of the sleeve. The user grasps the handle, bends the soft sheath to move the endoscope to the calyces. After the broken stones are found, the suction machine is started to suck out of the stones.

The use method of the endoscopic cannula is described as follows. The sheath 2 and the core rod 3 are combined. The location column 4 and the handle 1 are combined (or the location column 4 and the handle 1 are not combined; one person holds the location column 4 for water injection operation, the other holds the handle 1 to make the sheath 2 enter the human body and control the direction of the sheath; the two persons operate the handle 1 and the location column 4 separately, thus reducing the difficulty and improving the operation efficiency). The endoscope is inserted into the third channel 7 of the core rod 3 (optionally, the endoscope can be fixed in the third channel 7 in advance), and the lead wire is inserted into the first channel 5. The core rod 3 and the sheath 2 are guided by the lead wire to the operative site. The ureter can be observed under the endoscope, and when the obstacle occurs, the water can be injected into the second channel 6 to remove the obstacle. When the core rod 3 and the sheath 2 reach the operative site, the core rod 3 is extracted, a ureteroscope is inserted in the sheath 2 to perform flexible ureteroscopy. During the operation, the position of the ureteroscope can be adjusted by turning the steering mechanism 12, so that the soft and hard sheath can enter the renal pelvis and calyces and can be positioned. The ureteroscope, together with the operation instrument, breaks the stones in the renal pelvis and calyces. A vacuum machine is connected to the suction channel of the handle. When the broken stones are observed, the vacuum machine is started, and the front end of the ureteroscope exits from the soft sheath 2.2. The negative pressure in the soft sheath 2.2 will suck the broken stones out of the body. The broken stones are placed in the string bag 24. After the operation, the broken stones in the string bag 24 are treated. Based on the endoscopic cannula comprising the core rod 3 and the location column 4 of the disclosure, the ureteroscope can move to the operative site accurately. The operation is easy and convenient, can reduce the amount of bleeding and the pain of the patients, and improves the operation efficiency and success rate.

It will be obvious to those skilled in the art that changes and modifications may be made, and therefore, the aim in the appended claims is to cover all such changes and modifications.

What is claimed is:

1. A device, comprising:
1) a handle;
2) a sheath; and
3) a core rod disposed in the sheath and capable of sliding along an axial direction of the sheath;
wherein:
the core rod comprises a first channel, a second channel, and a third channel which are sequentially connected to one another;
the handle comprises a first joint configured to guide a lead wire into the first channel, a second joint configured to guide water into the second channel, and a third joint configured to guide an endoscope into the third channel; and
the sheath comprises a first end and a second end, and the second end is disposed in the handle.

2. The device of claim 1, wherein the sheath is a hard sheath; the hard sheath comprises a first end exposed out of the handle and a second end disposed in the handle; the hard sheath comprises an axial straight hole, and the core rod is in clearance fit with the axial straight hole.

3. The device of claim 1, wherein the sheath is a combination of a soft sheath and a hard sheath; the hard sheath comprises a first end exposed out of the handle and a second end disposed in the handle; the soft sheath comprises a first end and a second end, and the second end of the soft sheath is coaxial and integrated with the first end of the hard sheath; an inner diameter and an outer diameter of the soft sheath are the same as that of the hard sheath; the soft sheath and the hard sheath each comprise an axial straight hole, and the core rod is in clearance fit with the axial straight hole; the soft sheath is provided with a steering wire, and the handle comprises a steering mechanism on which the steering wire is fixed.

4. The device of claim 3, wherein the first end of the soft sheath is provided with a locking cap; the locking cap is coaxial with the soft sheath; one end of the steering wire is fixed on the locking cap, and the other end of the steering wire passes through the soft sheath and the hard sheath to fixedly connect to the steering mechanism.

5. The device of claim 4, wherein one side of the handle comprises a hand shank; the steering mechanism is disposed on the hand shank; and the hand shank comprises a plurality of grooves.

6. The device of claim 2, wherein the handle comprises a sleeve; the sleeve comprises a first end, a second end, and an axial through hole disposed between the first end and the second end; the axial through hole comprises a first section, a second section, and a third section; the hard sheath sequentially passes through the first section, the second section, and the third section and is fixed on the sleeve; the third section is a taper hole; the second end of the sleeve is provided with a location column, and the first joint, the second joint and the third joint are disposed on the location column.

7. The device of claim 3, wherein the handle comprises a sleeve; the sleeve comprises a first end, a second end, and an axial through hole disposed between the first end and the second end; the axial through hole comprises a first section, a second section, and a third section; the hard sheath sequentially passes through the first section, the second section, and the third section and is fixed on the sleeve; the third section is a taper hole; the second end of the sleeve is provided with a location column, and the first joint, the second joint and the third joint are disposed on the location column.

8. The device of claim 6, wherein the location column comprises a casing; the casing comprises a counter bore configured to receive the core rod; the counter bore comprises a first hole communicating with the first channel, a second hole communicating with the second channel, and a third hole communicating with the third channel; the first joint, the second joint and the third joint communicate with the first hole, the second hole, and the third hole, respectively; two sides of the casing each are provided with a connection plate and a fastener; two sides of the second end of the sleeve each are provided with a locating slot; and the locating slot matches the fastener in shape to lock the sleeve and the casing.

9. The device of claim 7, wherein the location column comprises a casing; the casing comprises a counter bore configured to receive the core rod; the counter bore comprises a first hole communicating with the first channel, a second hole communicating with the second channel, and a third hole communicating with the third channel; the first joint, the second joint and the third joint communicate with the first hole, the second hole, and the third hole, respectively; two sides of the casing each are provided with a connection plate and a fastener; two sides of the second end of the sleeve each are provided with a locating slot; and the locating slot matches the fastener in shape to lock the sleeve and the casing.

10. The device of claim 8, wherein the second end of the sleeve is provided with a disc; the disc is coaxial with the sleeve and comprises a via hole communicating and coaxial with the third section of the axial through hole; the disc comprises a circumferential flange, and the locating slot is disposed on the circumferential flange.

11. The device of claim 9, wherein the second end of the sleeve is provided with a disc; the disc is coaxial with the sleeve and comprises a via hole communicating and coaxial with the third section of the axial through hole; the disc comprises a circumferential flange, and the locating slot is disposed on the circumferential flange.

12. The device of claim 10, wherein two connection rods are connected to the circumferential flange, and one end of each of the two connection rods is provided with a string bag for accommodating broken stones.

13. The device of claim 11, wherein two connection rods are connected to the circumferential flange, and one end of each of the two connection rods is provided with a string bag for accommodating broken stones.

14. The device of claim 6, wherein one side of the sleeve is provided with a suction channel; the suction channel comprises a first opening communicating with the third section of the axial through hole and a second opening configured to connect to a vacuum machine; the first opening of the suction channel is disposed between the via hole and the first section of the axial through hole.

15. The device of claim 7, wherein one side of the sleeve is provided with a suction channel; the suction channel comprises a first opening communicating with the third section of the axial through hole and a second opening configured to connect to a vacuum machine; the first opening of the suction channel is disposed between the via hole and the first section of the axial through hole.

16. The device of claim 1, wherein the first channel shares one channel with the second channel, and the third channel is an independent channel.

17. The device of claim 1, wherein the first channel, the second channel, and the third channel of the core rod are independent from each other.
